(19) United States
(12) Patent Application Publication
(10) Pub. No.: US 2018/0327817 A1
(43) Pub. Date: Nov. 15, 2018

(54) Strain Typing Assay and Method Without Need for Isolation in Pure Form and Subsequent Longitudinal Strain Tracking

(71) Applicant: Rheonix, Inc., Ithaca, NY (US)

(72) Inventor: F. Morgan Wallace, Elkton, MD (US)

(73) Assignee: Rheonix, Inc., Ithaca, NY (US)

(21) Appl. No.: 15/978,333

(22) Filed: May 14, 2018

Related U.S. Application Data
(60) Provisional application No. 62/506,054, filed on May 15, 2017.

(51) Int. Cl. C12Q 1/689; C12Q 1/04; C12Q 1/6874; C12Q 1/6837; G06K 9/62

(52) U.S. Cl. CPC C12Q 1/689; C12Q 1/04; C12Q 1/6874; C12Q 1/6837; C12Q 2600/156; G06K 9/6255; G06K 9/6276; G06K 9/6292; G06K 9/6227

(57) ABSTRACT

A system and method for strain typing without need for isolation in pure form and subsequent longitudinal strain tracking. The method includes the steps of locating one or more genetic regions present in a target. The genetic regions contain genetic loci that vary among two or more variants (i.e., strains) of the target. A device detects a unique sequence of each of the genetic loci. After a sample of biological material having the genetic loci is obtained, the device generates an amplicon for the genetic regions present in the target. The amplicons are hybridized to complimentary probes, resulting in hybridized probes and non-hybridized probes, which are detected. The detected hybridized probes are assigned an identifier. The device transforms the identifiers into a pattern. The pattern is recorded and compared to one or more other patterns recorded to determine if the pattern is different from the one or more other patterns.

### CROSS-REFERENCE TO RELATED APPLICATION

The present application relates and claims priority to U.S. Provisional Application No. 62/506,054 filed May 15, 2017, the entirety of which is hereby incorporated by reference.

### BACKGROUND OF THE INVENTION

### 1. Field of the Invention

The invention relates generally to a system and method for nucleic acid analysis and more particularly, to a system and method of analyzing nucleic acid sequences from complex biological matrices and using the analysis for surveillance of the occurrence of such sequences.

### 2. Description of Related Art

Mixed biological populations may be studied to determine various attributes about the populations. Within any population there may exist subgroups at various levels of organization. The most common organizational structure within biological systems is the Linean System which may also be called the genus-species system. Using nucleic acid analysis, the Linean System may be refined to show a difference between individuals or differences between very closely related individuals within populations. For example, in certain organisms there may be only a difference in a single nucleic acid base in a single nucleic acid sequence that can be used to differentiate two subtypes of a single species in a population. Individuals within a population that vary by one or more nucleic acid bases in a genetic sequence are called sub-types or strains.

Identifying particular genus, species, or strains of organisms from a mixed population is an important activity. Most often, such studies require extensive, expensive, and time consuming protocols to isolate and purify the living organisms from one another in order to conduct such an identification or characterization. In many cases the purification takes too long, certain populations cannot survive the process, or the persistence of the living organism may be harmful to the technicians performing the analysis.

Current methods of strain typing require a pure culture of an organism before performing a molecular characterization of the organism. Such molecular characterizations may be conducted using sequence-based (including multi-locus or whole genome) approaches, macro-restriction digest (pulsed field gel electrophoresis) techniques or hybridization-based (automated or manual Ribotyping) methods. The processes of purifying a strain and performing the molecular characterization are both lengthy and expensive. Current methods are also perilous for various market segments. For example, food manufacturers or hospitals may incur significant liability if they identify an isolate that can be directly compared to an isolate from an ill person.

Further, the persistence of particular strains in the environment (either food manufacturing, agricultural, or health care setting) or in an individual environmental site, over time, can be indicative of failures of sanitation. Such persistence can lead to contamination of products manufactured or illness of animals or humans. Alternatively, the (random or cyclically) periodic occurrence of particular sequences provides information relating to repeated invasion of an environment by certain organisms.

Currently, testing instrumentation cannot pull and process the amount of the data (i.e., information) from a complex sample to provide such strain-related detail without first isolating the organism from the sample. In addition, conventional molecular diagnostic assays can only detect 3-5 targets in a single assay. This small number of targets is not enough information to provide sufficient strain discriminating power to be very useful.

Therefore, there is a need for a system and method for processing a complex (i.e., mixed) biological material from the environment and detecting the presence of particular strains of interest without specifically identifying an isolate.

### SUMMARY OF THE INVENTION

The present invention is directed to, inter alia, a system and method of analyzing nucleic acid sequences from complex biological matrices and using the analysis for surveillance of the occurrence of such sequences. In one embodiment, a method for analyzing genetic information comprises the steps of: (i) locating one or more genetic regions present in a target, wherein the genetic regions contain genetic loci that vary among two or more variants of the target; (ii) providing a device configured to detect a unique sequence of each of the genetic loci; (iii) obtaining a sample of biological material having the genetic loci; (iv) generating an amplicon for the one or more genetic regions present in the target; (v) hybridizing the amplicon to one or more probes for the genetic loci wherein the one or more probes will hybridize to a variant of the genetic loci and will not hybridize to a variant of the genetic loci; (vi) detecting, via the device, each probe hybridized to an amplicon; (vii) assigning an identifier to each hybridized probe which identifier is different from an identifier assigned to a non-hybridized probe; (viii) transforming, via the device, the assigned identifiers for each probe into a pattern of identifiers of the hybridized probes which is recorded; (ix) comparing the pattern recorded to one or more other patterns recorded; and (x) determining if the pattern recorded is different from the one or more other patterns recorded.

In another embodiment, a method for strain-typing a target organism in a complex biological material, comprises the steps of: (i) amplifying nucleic acid sequences that contain variable genetic loci from the complex biological material, via a device, to generate an amplicon; (ii) hybridizing the amplicon to one or more probes for the genetic loci, wherein the one or more probes will hybridize to a variant of the genetic loci and will not hybridize to a variant of the genetic loci, on at least one of a first hybridization array and a first bead; (iii) detecting the one or more hybridized probes and the one or more non-hybridized probes on the at least one of the first hybridization array and the first bead; (iv) assigning an identifier to each hybridized probe and non-hybridized probe on the at least one of the first hybridization array and the first bead; (v) generating a first pattern of one or more identifiers on the at least one of the first hybridization array and the first bead; (vi) comparing the first pattern of the at least one of the first hybridization array and the first bead to a second pattern of at least one of a second hybridization array and a second bead; and (vii) determining if the first pattern is different from the second pattern.

### BRIEF DESCRIPTION OF THE DRAWINGS

One or more aspects of the present invention are particularly pointed out and distinctly claimed as examples in the claims at the conclusion of the specification. The foregoing and other objects, features, and advantages of the invention are apparent from the following description taken in conjunction with the accompanying drawings in which:

FIG. 1 is a top view of an illustrative embodiment of a consumable device used in the workstation of the system of the prior art;

FIG. 2 is a flowchart of an illustrative embodiment of a method for analyzing nucleic acid sequences from complex biological matrices and using the analysis for surveillance of the occurrence of such sequences;

FIG. 3 is a diagram of an illustrative embodiment of an alignment between nucleic acid sequences which may be used to select a target for preparation of hybridization arrays or beads and the primers required to generate the amplicons to bind to particular nucleic acid sequences on the array or bead;

FIG. 4 is a diagram of an illustrative embodiment of a differentiation scheme using beads for strain characterization;

FIG. 5A is a diagram of an illustrative embodiment of a simplified three loci differentiation scheme using a hybridization array for strain characterization;

FIG. 5B is an additional diagram of an illustrative embodiment of simplified three target differentiation scheme using a hybridization array for strain characterization;

FIG. 6A is a top view of an illustrative embodiment of a 9 by 3 hybridization array with all of the available variant genetic loci probe spots hybridized to amplicons and three spots used for orientation of a camera to record the pattern of spots;

FIG. 6B is a top view of an illustrative embodiment of a 9 by 3 hybridization array from a biological material showing only seven variant genetic loci probe spots hybridized to amplicons and three spots available for orientation of a camera to record the pattern of spots, and a table representing the conversion of the spot locations into a binary code representing the pattern of hybridization events where the amplicons have hybridized to their complimentary probe located at a known position on the array;

FIG. 7 is a top view of an illustrative embodiment of a pair of hybridization arrays with the same positional arrangement of probes and camera orientation spots as in FIG. 6A and FIG. 6B generated from two separate biological materials which show the same pattern of amplicon hybridization on both arrays;

FIG. 8 is a top view of an illustrative embodiment of a pair of hybridization arrays with the same positional arrangement of probes and camera orientation spots as in FIG. 6A and FIG. 6B generated from two additional separate biological materials which show different patterns of amplicon hybridization on the two arrays;

FIG. 9 is a timeline of a representative current method of strain typing Listeria used by food safety regulators and epidemiologists compared to a timeline of an illustrative embodiment of a method for strain typing Listeria without the need for isolation in a pure culture, including also a simplified report comparing the patterns generated used to compare pattern results for recurrence or uniqueness;

FIG. 10 is a chart of an illustrative embodiment of a method for strain typing six Listeria strains including a filter key detailing the probe locations and probe types on an illustrative 9 by 3 array, and an alternative pattern coding procedure;

FIG. 11 is a top view of hybridization arrays generated from a 6 different Listeria strains analyzed first from pure culture and second spiked into a complex environmental enrichment; and

FIG. 12 is a top view of hybridization arrays generated from 12 different Listeria strains each analyzed from a spike negative environmental enrichment.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

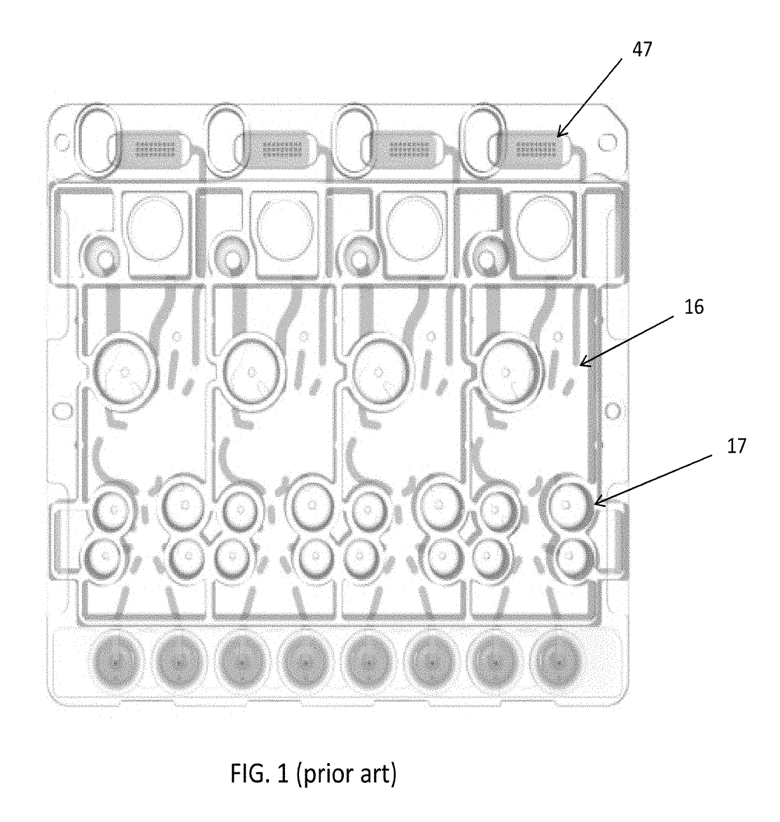

The present invention is a system and method for analyzing the occurrence of patterns of nucleic acid sequences from complex biological matrices and using the analysis for surveillance of the duplication or variation of the patterns generated from the analyses of such sequences. The system may comprise a conventional workstation including consumables, such as the cartridge used in the Rheonix Optimum™ workstation shown in FIG. 1, for example, and a test kit containing reagents therefore (not shown). Exemplary structural and functional aspects of embodiments of the present invention are similar to or include elements of the workstation and its consumables, described and illustrated in U.S. Pat. No. 8,383,039. Those similarities should be understood by a person of ordinary skill in the art in conjunction with a review of this disclosure and accompanying drawings in conjunction with the published patent, and are not further discussed in detail herein. The (prior art) top view of a consumable device (e.g., cartridge) shown in FIG. 1 interfaces with the workstation and is where an exemplary version of the assay is performed. The device contains a fluid reservoir layer 17 which contains reservoirs connected to truncated channels 16 formed in the bottom of the fluid reservoir layer 17. Certain reservoirs in the fluid reservoir layer 17 contain low density nucleic acid probe arrays 47 for hybridization of amplicons to the arrayed probes. The Rheonix Optimum™ workstation coupled with a cartridge, such as that shown in FIG. 1, performs the steps of the method described herein in an automated fashion. The system and method may also be performed using other means in the place of the hybridization array such as beads each containing a unique identifier and coupled each to their own oligonucleotide probe such that an analysis of the hybridization events may be accomplished by analyzing each bead and generating a pattern commensurate with the patterns generated herein.

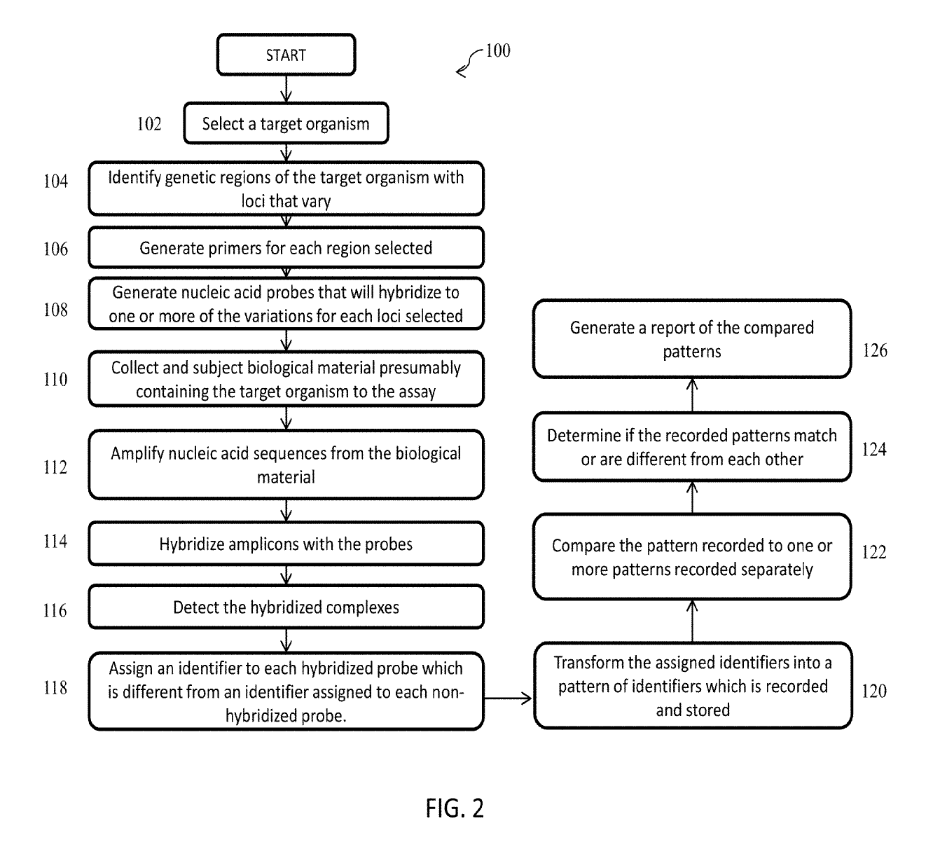

Turning now to FIG. 2, there is shown a flowchart of a method 100 for analyzing nucleic acid sequences from complex biological matrices and using the analysis for surveillance of the duplication or variation of the patterns generated from the analyses of such sequences. At the first step 102, a target organism for surveillance is identified. Such target organism should have at least two genetic regions that are unique to the target organism and that vary among the strains of the target organism. In order to perform the method directly from a sample containing a large background of non-target flora, the selection of genetic regions that are unique to the target organism are critical since the unique regions allow the assay to only amplify the genetic regions present from the target organism even in a background of an excess of non-target genetic material.

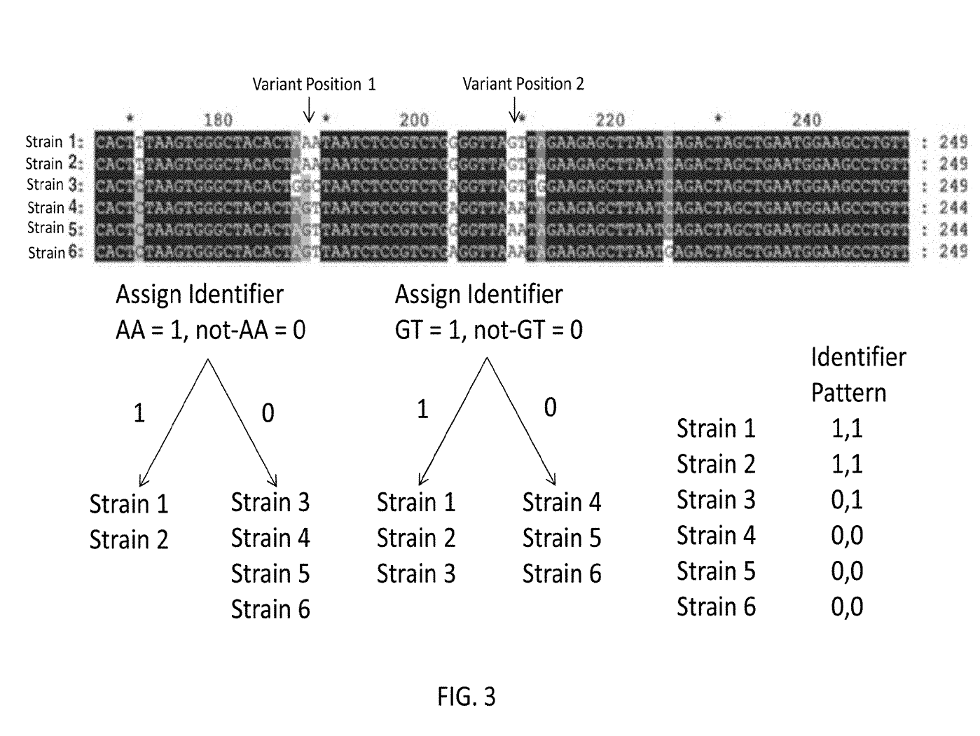

At the following step 104, genetic loci within the identified genetic regions of the target organism are identified. In the embodiment shown in FIG. 3, within the genetic regions shown, there are two genetic loci variants shown at Variant Position 1 and Variant Position 2 for 6 strains of the target organism (i.e., base pairs at Variant Position 1 and Variant Position 2 along the nucleic acid sequences are different among the 6 strains). As shown in FIG. 3, each potential outcome of a hybridization event is assigned an identifier, such as a binary digit, for example. As shown in FIG. 3, a base pair at the first position of each of the 6 variants may be AA, GC or GT. In the example, base pairing AA is assigned the binary digit “1”, while any other base pairing termed “not AA” is assigned the binary digit “0”. Similarly, a base pair at the second position of each of the 6 variants may be GT or AA. In the example base pairing GT is assigned the binary digit “1”, while any other base pairing termed “not GT” is assigned the binary digit “0”. Thus, with two loci that exhibit variation using a binary system there is a total of four possible binary code combinations for each nucleic acid sequence (“0,0” or “0,1” or “1,0” or “1,1”). In the embodiment shown in FIG. 3, there are six strains of a target which generate three of the four possible binary code combinations. For example, the first strain has both the AA base pair at the first position and the GT base pair at the second position; therefore, the first strain has the binary code of “1,1”. On the other hand, the sixth strain has the GT base pair at the first position and the AA base pair at the second position; therefore, the sixth strain has the binary code of “0,0”. The example further shows that among the six strains, three patterns are generated. There are two “1,1” patterns, one “0,1” pattern, three “0,0” patterns and zero “1,0” patterns. Importantly the patterns generated do not differentiate strains 1 and 2 from each other, but they do differentiate strains 1 and 2 from the other 4 strains. While strains 4, 5 &6 are also not differentiated from each other, they are differentiated from strains 1, 2 & 3. In practice, a selection of n loci which exhibit such variation will provide up to 2ⁿ (where n=the number of positions) potential patterns. For example, the 2 loci of the example generate 4 patterns, 3 loci generate 8 patterns, 4 loci generate 16 patterns, 5 loci generate 32 patterns and 6 loci will provide 64 potential hybridization patterns and so forth for as many loci for which probes are prepared. Selection of the particular variant loci and probes must be such that the probes will sort the variants into small enough groupings (each grouping representing a pattern). In one embodiment, groupings that are small enough have at most 35% of the target organisms sorting into any one group. However, the groupings must also be large enough that each variant (i.e., strain) is not sorted into its own group. In other words, the loci and probes must be selected such that a single pattern represents, at most, 35% of the target organisms, but not any one particular strain of the target organism.

As shown in step 106, after a variant position is selected, nucleic acid primers are generated to perform an amplification reaction to generate amplicons of the sequences containing the variant positions. Nucleic acid probes are generated at the next step 108 and are designed to hybridize to certain of the amplicons and not to hybridize to others of the amplicons, in the manner described in FIG. 3, to generate binary outcomes for each position. Detection of a targeted nucleic acid sequence requires the use of a nucleic acid probe having a nucleotide base sequence that is substantially complementary to the targeted sequence or, alternatively, its amplicon. Under selective assay conditions, the probe will hybridize to the target sequence or its amplicon in a manner permitting a practitioner to detect the presence of the target sequence when present in the sample. Effective probes are designed to prevent nonspecific hybridization with any nucleic acid sequence that will interfere with detecting the presence of the targeted sequence. Probes and/or the amplicons may include a label capable of detection, where the label is, for example, a radiolabel, fluorescent dye, biotin, enzyme, electrochemical or chemiluminescent compound. In some embodiments the hybridization may be to a probe immobilized on a bead in which case the bead may also have a particular label such that the bead itself is identified or a combination of identifying the bead and a label on the oligonucleotide is used. In an embodiment of the method described herein, the presence or absence of the target sequences is detected by a camera on the workstation using reverse dot blot (RDB) hybridization. A camera on the workstation captures an image of the resultant hybridization array and under the control of software which implements a gray scale image processing procedure selects the hybridization spots that are dark enough to represent successful hybridization events or are not dark enough to represent successful hybridization events. The gray scale values are preset using data generated during assay development. The successful hybridizations are then given an identifier which may be a “1” or some other unique identifier for the position on an array. The unsuccessful hybridizations are given an identifier which may be a “0”.

At the next step 110, a source of biological material is subjected to the assay. Such biological material can be in a native state such that the organisms contained therein are not isolated one from another. In one embodiment, the biological material is from a pure culture. In another embodiment, the biological material is from a complex enrichment. When performing a nucleic acid-based assay, preparation of the sample is the first and most critical step to release and stabilize nucleic acids that may be present in the sample. Sample preparation can also serve to eliminate nuclease activity and remove or inactivate potential inhibitors of nucleic acid amplification or detection of the nucleic acids. The workstation of the system performs all of the sample preparation steps in an automated fashion with only a single technician-performed (i.e., user-performed) pipetting step needed. Utilizing the test kit and the workstation, the user can prepare the sample by carrying out cell lysis and nucleic acid purification (i.e., DNA isolation). In another embodiment of pattern typing without the need to fully isolate and purify the target organism in pure culture, the preparation of the sample includes a preliminary immunomagnetic separation (IMS) performed either on the workstation or off-line to remove cross-reactive species. For example, a preliminary IMS may be required for particular target organisms, such as Salmonella.

At the following step 112, after nucleic acid (e.g., DNA) isolation, the workstation, without any additional input from the user, transfers the purified nucleic acid to reaction reservoirs where amplification of specific nucleic acid sequences occurs. Particular genetic sequences from the biological material are amplified to obtain the nucleic acid sequences of the biological material. Specifically, nucleic acid amplification is the enzymatic synthesis of nucleic acid amplicons (i.e., copies) which contain a sequence that is homologous to a nucleic acid sequence being amplified. Examples of nucleic acid amplification procedures practiced in the art include the polymerase chain reaction (PCR), strand displacement amplification (SDA), ligase chain reaction (LCR), Nucleic Acid Sequence Based Amplification (NASBA), transcription-associated amplification (TAA), Cold PCR, and Non-Enzymatic Amplification Technology (NEAT), among others.

Nucleic acid amplification is especially beneficial when the amount of target sequence present in a sample is very low. By amplifying the target sequences and detecting the synthesized amplicons, the sensitivity of an assay can be vastly improved because fewer target sequences are needed at the beginning of the assay to better ensure detection of nucleic acid in the sample belonging to the organism or virus of interest. In an embodiment of the method described herein, sequences specific for the target organism with polymorphisms between strains are amplified. In other words, amplification of the sequences which are specific to the target organism, but which also contain enough differences that both detection and strain characterizations are possible. That is, sequences are selected that are specific for the target organism (not present in other genera or species) but not present in 100% of strains of the target genera or species. Enough of these sequences are selected and amplified such that strains of the target organisms can be differentiated.

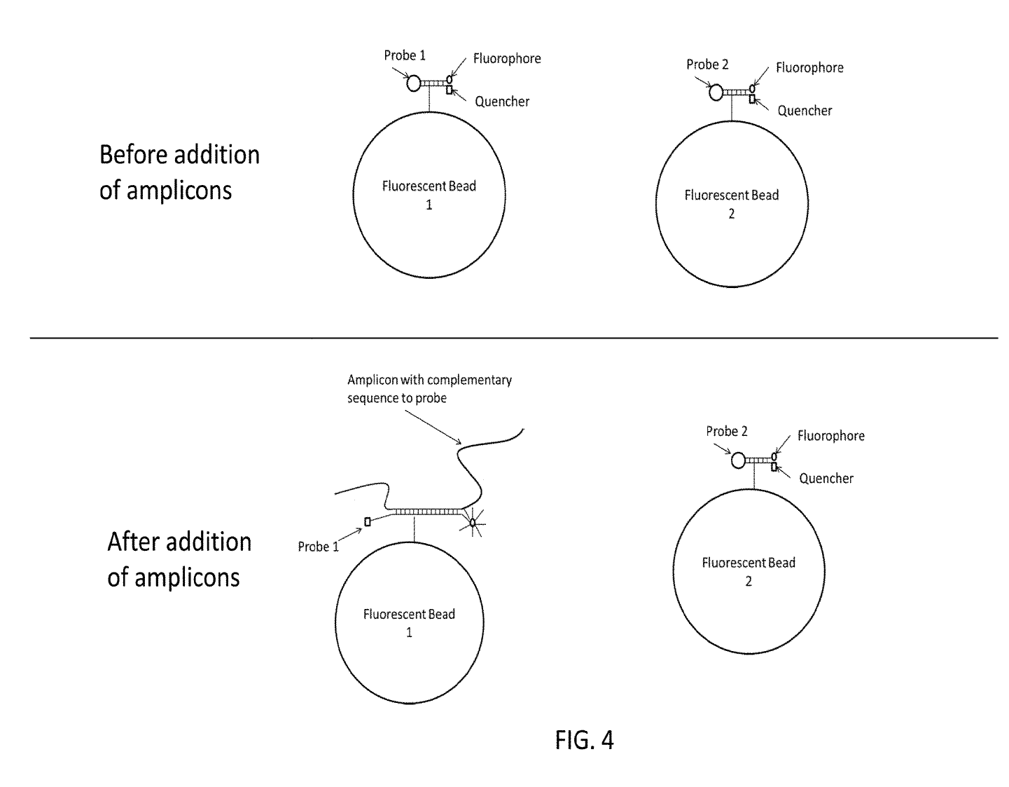

Referring now to FIG. 4, there is shown a bead-based format. In the bead-based format of the depicted embodiment, each variation of a probe can be added to a distinct fluorescently-labeled bead. The beads are used for detecting binding events by illuminating the beads and analyzing them with a detector for both the bead fluorescent characteristics and the nucleic acid probe linked fluorophore characteristics to determine which amplicon variants are present. In this case, Fluorescent Bead 1 would give a signal for both its bead and the probe's fluorophore (a positive result), while Fluorescent Bead 2 would give a signal for only the bead, and not a signal for its probe's fluorophore (a negative result) because no complimentary amplicons were present. Many types of bead/probe combinations can be highly multiplexed to form a pattern-based typing scheme as described herein.

Ultimately, the analysis of the hybridization of the amplicons generated from the biological material described above is conducted with a system with enough multiplexing capability such that at least 6 hybridization probes (i.e. at least 64 patterns) can be analyzed to determine presence or absence of specific hybridizations of the amplicons generated from the biological material. As shown in the examples above, the presence of hybridization of the amplicons with the predetermined probe sequences may be obtained using colorimetric, fluorimetric, radiographic, electrophoretic, mass spectrographic or any other such identifying analytical methodology which can provide an absent/present hybridization determinant for each of the probe sequences.

For example, at the next step 114 of the embodiment of the method described herein, amplicons are captured (i.e., hybridized) by their complimentary probes. After probe capture, at the following step 116, the hybridized probes (and non-hybridized probes) are detected. In one embodiment a camera on the workstation detects bound DNA by imaging darkening of a reporter molecule that is deposited due to an enzymatic activity bound to the amplification product on an array. If no amplification product is manufactured (i.e., a non-hybridized probe), there is no darkening of a given spot. In another embodiment, beads with hybridized probes may be detected and analyzed with a suitable system. At the next step 118, the system assigns an identifier to each probe location based on the gray scale cut off value of the imaging software as describe above. At the next step 120, the identifiers are transformed into a pattern of identifiers and the pattern is recorded and stored.

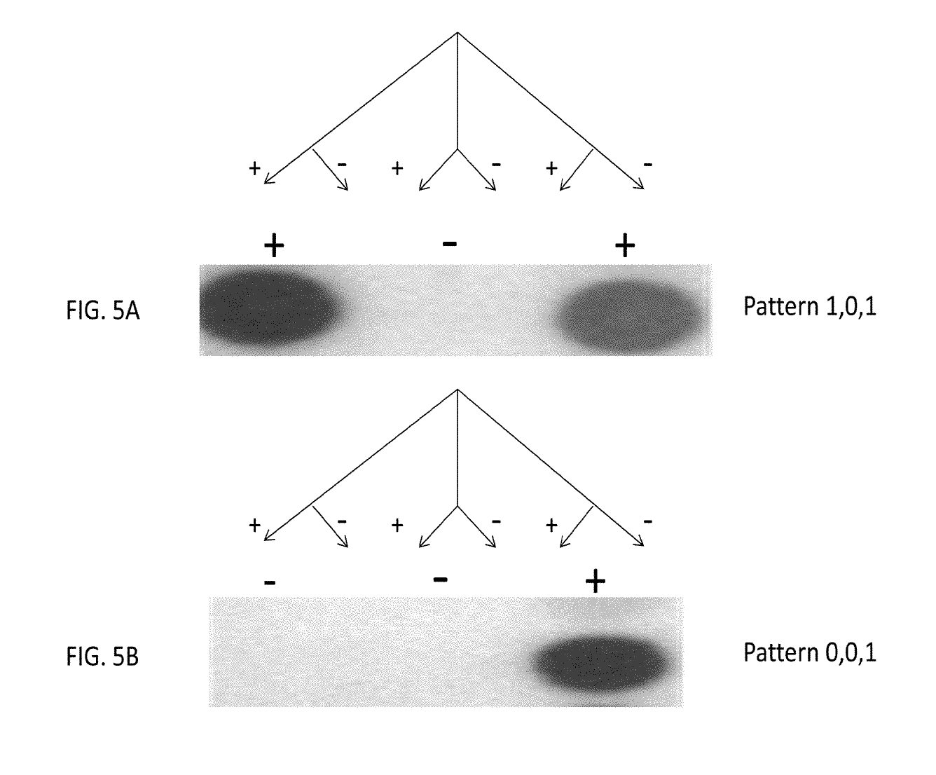

Turning now to FIGS. 5A and 5B, there are shown diagrams of an illustrative embodiment of a simplified three loci differentiation scheme for strain characterization. In the depicted embodiment, there are three loci (or positions of base pairs which is one more than is shown in FIG. 3, providing up to 8 possible patterns of probe hybridization) that distinguish strains of the target organism. As described above, when there is hybridization of the probe with the amplicon, the binary digit “1” is assigned. Similarly, when there is no hybridization it is assigned the binary digit “0”. For example, as shown in FIG. 5A, the binary code pattern is “1, 0, 1” because the assay shows two dark spots at the first probe position and third probe position, and no spot (thus, no hybridization to the probe) in the second probe position. In another example, as shown in FIG. 5B, the binary code pattern is “0, 0, 1” because the assay shows one dark spot at the third probe position and no spots (thus, no hybridization to the probes) in the first and second probe positions.

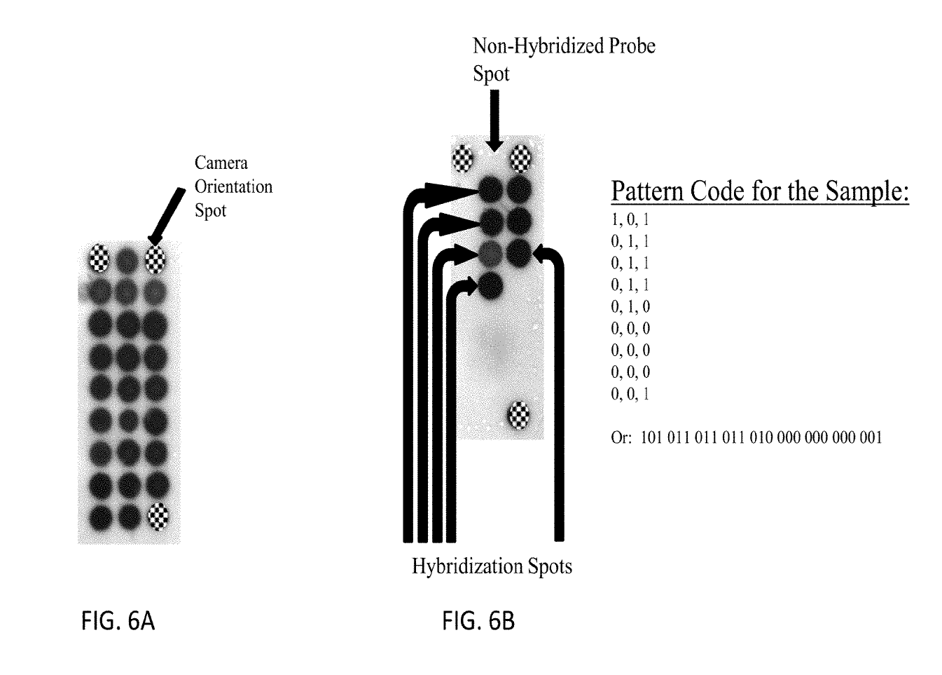

Referring now to FIGS. 6A and 6B, there are shown top views of illustrative embodiments of 9 by 3 hybridization arrays. The hybridization array of FIG. 6A shows all available probes hybridized to amplicons and comprises an arrangement of reference spots (shown in a circular checkerboard pattern) amongst the dark spots. The reference spots are used to orient the camera of the workstation for correct image capture and to help verify that the assay was performed properly. The camera utilized is a conventional camera mounted in a workstation similar to that described and illustrated in U.S. Pat. No. 8,383,039. Those similarities should be understood by a person of ordinary skill in the art in conjunction with a review of this disclosure and accompanying drawings in conjunction with the published patent, and are not further discussed in detail herein. The array on the hybridization membrane is arranged in a 3 column and 9 row format so that the particular spots are always in the same place relative to the camera reference spots. The image captured by the camera is subjected to a software program run on the workstation (or run on a device connected to the workstation) and designed to characterize the gray scale of each of the particular spots. The software is provided particular values of gray scale, above which, the software assigns an identifier (such as a “1”), indicating a successful hybridization—and below which, it assigns a different identifier (such as a “0”), indicating unsuccessful hybridization. All of the spots in FIG. 6A are hybridized and would be assigned the identifier “1”.

Turning now to FIG. 6B, there is shown a representative hybridization membrane derived from a sample of biological material. At step 120 of the method, the identifiers assigned to the probe positions with either successful or unsuccessful hybridizations are transformed into a pattern (i.e., code) of identifiers for the biological material and the pattern is recorded and stored. FIG. 6B, which illustrates such a pattern, shows the same camera orientation spots as shown in FIG. 6A; however there are fewer dark, positive spots, which indicates a hybridization (or binding) event. The probes are arranged in the same row and column format as used in FIG. 6A. The binding events (i.e., dark spots) are assigned binary digit “1” and the non-binding events (i.e., no spots) are assigned binary digit “0”. The assigned “l's” and “0's” generate a binary code when read across each row and the array. The resulting binary code represents all the hybridizing and non-hybridizing events from a particular sample. In the embodiment depicted in FIG. 6B, the first row has the binary code “1, 0, 1” with the “l's” representing the control spots. The binary code for the second row has the binary code “0, 1, 1” with the “l's” representing the dark, positive spots in the second and third columns. Thus, the pattern (i.e., code) for the biological material may be read from left to right and top to bottom. FIGS. 6A-6B show an array with 24 available probes since it is a 3 column 9 row array, providing 27 probes less the 3 probes used for orientation. Thus, the array in FIGS. 6A-6B provides up to 2²⁴ or 16, 777,216 potential patterns.

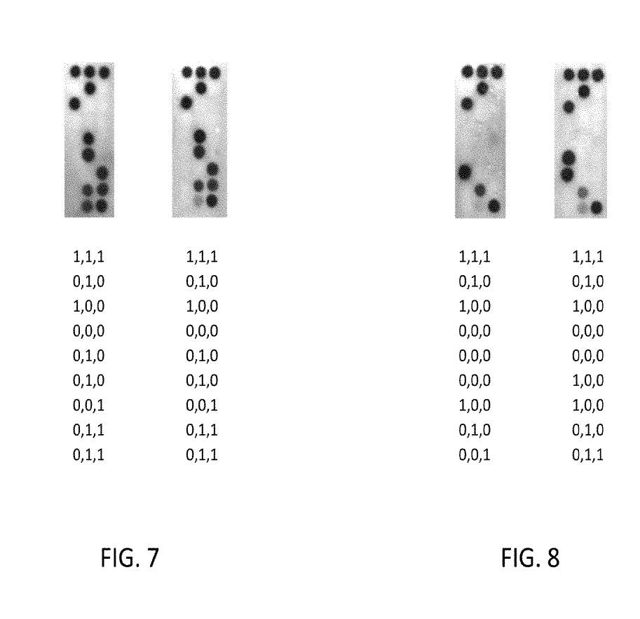

Referring now to FIGS. 7 and 8, there are shown top views of illustrative embodiments of hybridization arrays generated from biological materials. At the following step 122 of the method, the patterns (i.e., codes) for one or more hybridization arrays are compared. FIG. 7 shows a pair of hybridization arrays generated from two separate biological materials. The binary code (i.e., pattern) generated for each of the samples is shown below the corresponding hybridization array for each sample. The pattern for the each biological material in FIG. 7 is the positive spots in the array, indicating hybridization events. Identical binary codes between two separate biological materials indicate that the samples contained the same set of variants. The sample may be of the same strain or from two different strains that report the same pattern (for example, see strains 1 and 2 of FIG. 3). Persistence of a particular pattern generated from two or more samples may indicate that one or more strains is in a population that is not changing. In a food-production environmental sampling program, results from pattern recognition assays developed using this invention can be used to inform modifications to sanitation standard operating procedures (SSOPs) to reduce the risk of persistent potential finished product contaminating organisms. These organisms can be either pathogens that can lead to outbreaks, or quality organisms that can lead to economic losses associated with food spoilage.

Turning now to FIG. 8, there is an additional pair of separate biological materials (separate from those shown in FIG. 7 as well). The binary codes (i.e., patterns) for the separate biological materials shown in FIG. 8 are not identical. The difference in patterns is evidenced by the difference in location of the dark, positive spots, indicating a hybridization event, on the arrays. Different patterns generated between two separate biological materials indicate that different sets of variants are present in the separate samples and the transient nature of one or more strains or populations. Thus, the next step, step 124 of the method, is determining if the patterns match or are different, and is thereby characterizing the biological materials as persistent or transient. In other words, by comparing the patterns, it can be determined if one or more strains or populations of a target organism (e.g., Listeria) is present in one or more biological materials.

After the determination (of step 124) for these patterns for each biological material analyzed, as shown in FIG. 6A-8, the final step 126 includes generating a useful report which can be stored in a suitable computational system, database, or any other suitable storage media for later comparison to additional analyses of biological materials. The comparison between the repository of past stored patterns and newly obtained patterns provides for a method of identifying similarities and differences between the samples of biological material. Such comparison is important for detecting pathogens in numerous fields. For example, if a recurring pattern from longitudinally collected food manufacturing facility environmental, primary production or food samples is found (e.g., a pattern which is shown to repeat, as shown in FIG. 7), it is likely that the target organism is a persistent population. If, on the other hand, different or transient patterns are found to be present, as shown in FIG. 8, from longitudinally collected samples, the target organisms are likely to be different populations and to originate from separate reservoirs.

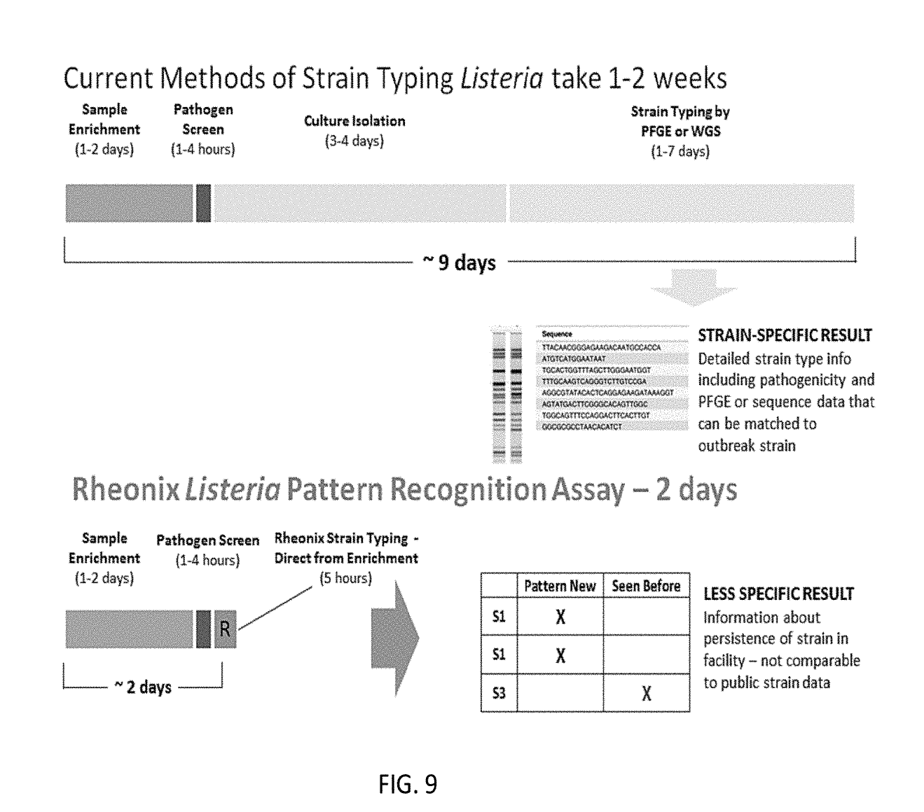

Turning now to FIG. 9, there is shown a timeline of a current method of strain typing Listeria compared to a flowchart of an illustrative embodiment of a method for strain typing Listeria from a complex enrichment without the need for isolation in a pure culture. In summary, the current method for strain typing Listeria begins with the step of enriching a sample, which takes approximately 1-2 days. Next, a molecular diagnostic screening test is performed over the course of a couple hours. Thereafter, culture isolation is performed, which takes approximately 3-4 days. Finally, molecular strain typing is performed on the culture over the course of 1-7 days. Therefore, the current method's total timeline for strain typing Listeria takes approximately 5-13 days.

Still referring to FIG. 9, the current method of strain typing Listeria is compared to an illustrative embodiment of the present invention. The method for strain typing Listeria from a complex enrichment without the need for isolation in pure culture dramatically reduces the time required to complete the strain typing. Similar to the current method, the illustrative embodiment requires that the sample be enriched, taking approximately 1-2 days. Also, molecular diagnostic screening tests are conducted over the course of 1-4 hours thereafter. However, where the present invention outperforms the current method for strain typing is in the final step. According to the illustrative embodiment shown in FIG. 9, the strain typing can be performed directly from the enrichment in up to 5 hours. Thus, the current method of culture isolation and molecular strain typing therefrom taking 4-11 days in condensed into 5 hours using the present invention. Ultimately, the illustrative embodiment of the method for strain typing from a complex enrichment without culture isolation takes approximately 2-3 days compared to the current method, which takes 5-13 days. FIG. 9 also shows a simplified three-sample report indicating that Sample 1 (S1) and Sample 2 (S2) are new unique patterns while Sample 3 (S3) is a previously generated pattern.

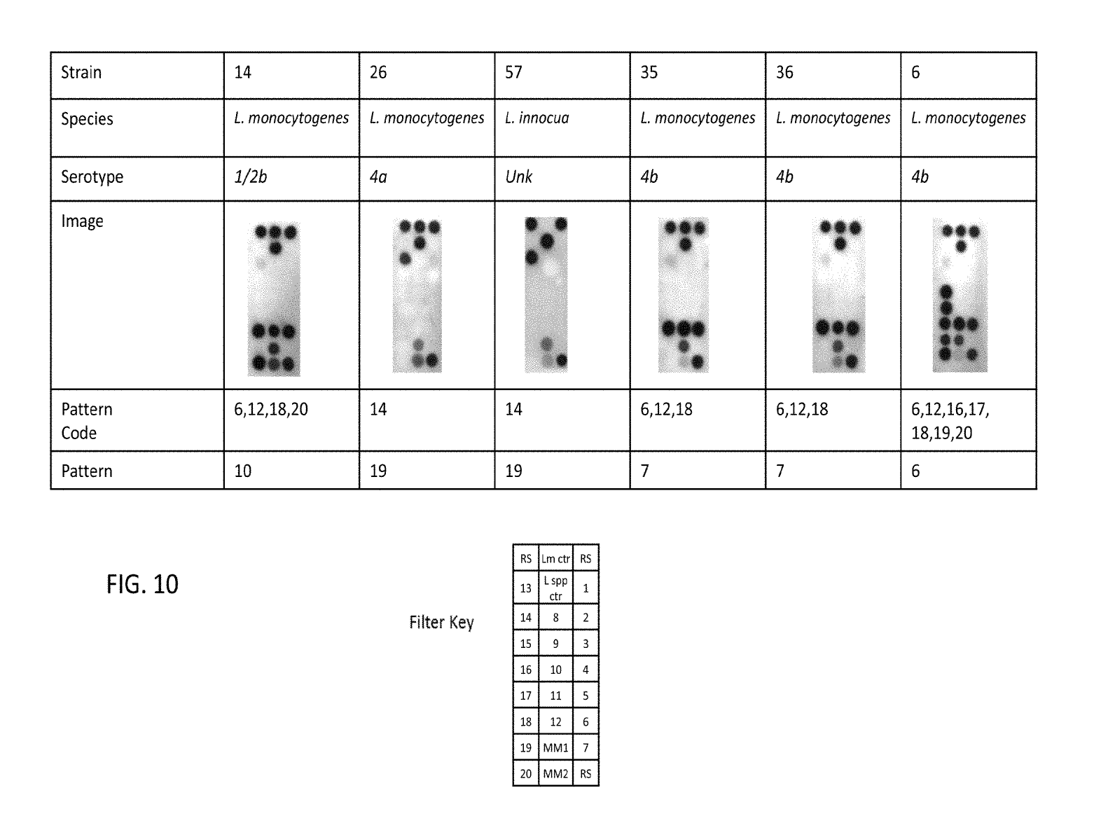

Referring now to FIG. 10, there is shown a chart of an illustrative embodiment of a method for strain typing six Listeria strains. The chart shows two different species for a total of six different strains of Listeria. The Serotype row denotes results from a traditional strain typing method. As shown in the strain row, the present invention can distinguish two different strains that are considered the same using the Serovar method (Pattern 7). Hybridization assays using the system and method of the present invention are shown in the row below the Serotype results.

As shown in FIG. 10, the hybridization assays resulting from the system and method of the present invention show four patterns (6, 7, 10 & 19). Two patterns (7 & 19) are the same pattern for separate strains. The patterns are transformed using the “Filter Key” shown. The Filter Key shows the camera orientation spots (or Reference Spots “RS”), two assay control spots (MM1 & MM2), two species identification spots (Lm ctr & L spp ctr) and assigns numeric values to each other potential spot location on the hybridization array. By comparing (generally performed by the camera and the image processing software) the hybridization arrays in to the Filter Key, the arrays are transformed into a numeric code. For example for strain four the arrays is transformed into the pattern code 6, 12, 18, 20, which is further transformed into pattern number 10. There are up to 2²⁰ or 1,048,576 possible patterns that may be generated from the 20-spot array shown in this example.

Users of the system receive numeric codes (i.e., patterns) or reports thereof generated for each biological material tested. Based on the patterns, the user can determine whether the biological materials have the same population of strains of Listeria or dissimilar populations of strains of Listeria. If the user continues to see the same pattern upon testing multiple biological materials from the same or a variety of locations, the user knows that the repeating pattern represents the same populations of strains of Listeria. With only the numeric codes, the user has enough knowledge to make rapid science-based changes to their sanitation standard operating procedures (SOPs) to assist with producing safe finished products.

Receiving only the pattern is beneficial to the user because it decreases the user's exposure to liability that occurs with other subtyping methods. In particular, the U.S. Food and Drug Administration (FDA) requires reporting of particular strains of Listeria. The FDA then makes the reported Listeria presence publicly known, shuts down production and other activities at the location of the reported strain, and requires a variety of compliance measures on behalf of the user. Therefore, the numeric code provides enough information for the user to know if there is a resident population of strains or transient populations of strains without knowing the particular strain, limiting the user's exposure to enhanced FDA regulations.

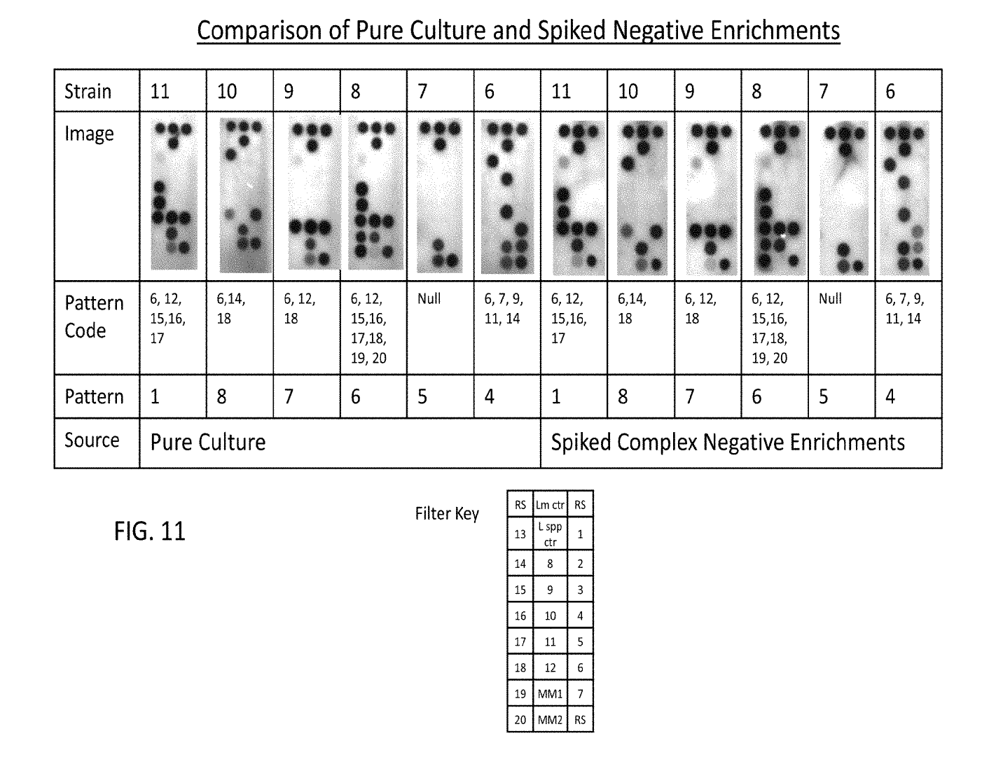

Turning now to FIG. 11, there is shown a top view of hybridization arrays generated from comparing the performance of the assay between Listeria strains from pure culture to Listeria strains from a complex environmental enrichment. The two sets of hybridization arrays shown in FIG. 11 have identical patterns for each particular strain shown either from pure culture or from complex enrichment. For the complex enrichment a Listeria strain was artificially introduced into a pre-enriched Listeria negative environmental enrichment. Specifically, the same Listeria replicates from the pure culture sample were inoculated into environmental samples that were pre-enriched and found to be negative for the presence of the target organism.

The spiked environmental enrichments were analyzed using the system and method of the present invention. As a result, the hybridization arrays detect and strain-type Listeria which occurred in a mixture of different organisms from the environment. FIG. 11 is evidence that the system and method of the present invention produces the same pattern when the isolate used is tested in pure culture and when a biological material is taken from the environment. Thus, FIG. 11 confirms that the culture isolation and molecular strain typing currently and routinely performed by the current method is no longer required, significantly decreasing the time it takes to strain-type a biological material.

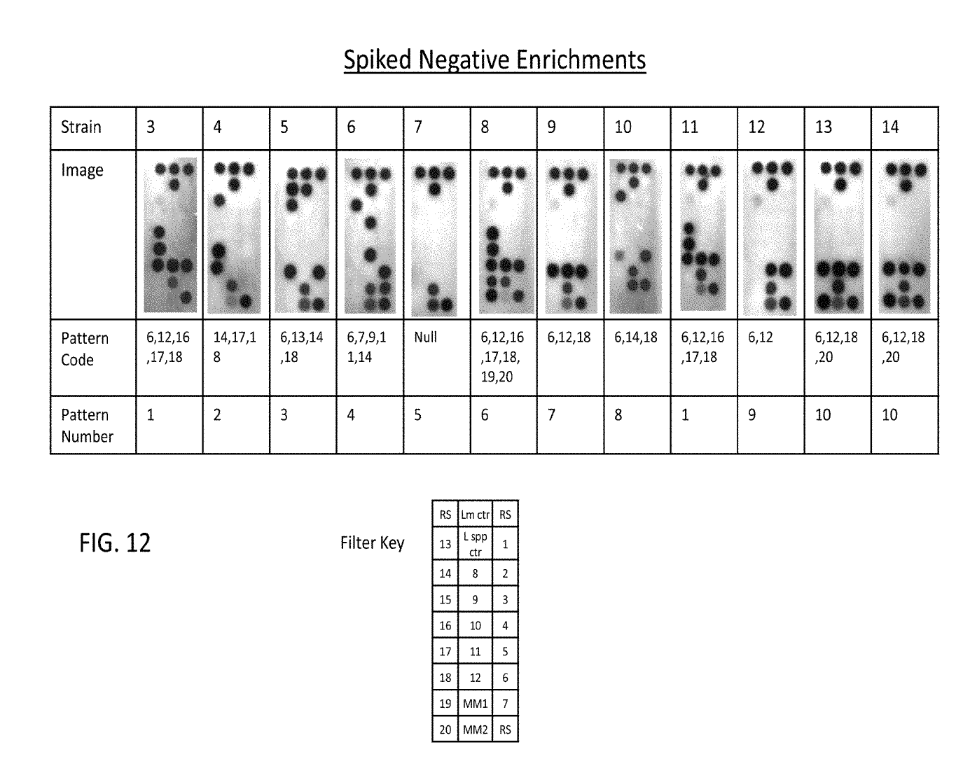

Now turning to FIG. 12, there is shown a top view of hybridization arrays generated from twelve Listeria strains spiked into pre-enriched Listeria negative environmental enrichment. The arrays confirm that the method herein can sort the twelve strains into 10 separate patterns. Note that the strains used in FIG. 11 are repeated again in FIG. 12 with six additional strains added. The arrays show that a careful selection of the genetic region from which the particular set of variable loci are determined provides a robust method of sorting the population of Listeria resident in the population into actionable information based on patterns generated from assaying the loci using the method herein. As the Listeria present in the enriched sample do not need to be isolated in pure culture before performing molecular subtyping, the results can be used to inform decisions regarding sanitation protocols much more quickly than currently available methods. This method will also significantly reduce the cost of performing molecular subtyping as a component of an environmental monitoring program and thus will make advanced molecular strain characterization available to a wider range of food producers.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

The recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A method for analyzing genetic information, comprising the steps of:
a. locating one or more genetic regions present in a target, wherein the genetic regions contain genetic loci that vary among two or more variants of the target;
b. providing a device configured to detect a unique sequence of each of the genetic loci;
c. obtaining a sample of biological material having the genetic loci;
d. generating an amplicon for the one or more genetic regions present in the target;
e. hybridizing the amplicon to one or more probes for the genetic loci wherein the one or more probes will hybridize to a variant of the genetic loci and will not hybridize to a variant of the genetic loci;
f detecting, via the device, each probe hybridized to an amplicon;
g. assigning an identifier to each hybridized probe which identifier is different from an identifier assigned to a non-hybridized probe;
h. transforming, via the device, the assigned identifiers for each probe into a pattern of identifiers of the hybridized probes which is recorded;
i. comparing the pattern recorded to one or more other patterns recorded;
j. determining if the pattern recorded is different from the one or more other patterns recorded.

2. The method of claim 1, wherein there is a minimum of six genetic loci present in a target.

3. The method of claim 1, wherein the biological material is a pure culture.

4. The method of claim 1, wherein the biological material is a complex mixture.

5. The method of claim 1, wherein the pattern and one or more other patterns are stored in a database connected to the device via at least one of a wired connection or a wireless connection.

6. The method of claim 1, further comprising the step of comparing the pattern stored in the database to a prior pattern stored in the database, wherein the prior pattern was obtained from a previously analyzed sample.

7. The method of claim 1, further comprising the step of reporting the pattern, wherein the pattern represents a set of defining genetic characteristics of the biological material.

8. The method of claim 1, wherein the target is Listeria.

9. The method of claim 8, wherein the two or more variants of the target are strains of the Listeria.

10. The method of claim 1, wherein the pattern is a series of two or more binary digits.

11. The method of claim 1, wherein the sample is a biological material from the environment.

12. The method of claim 1, wherein the step of hybridizing the amplicon to one or more probes for the genetic loci is conducted on a hybridization array.

13. The method of claim 1, wherein the step of hybridizing the amplicon to one or more probes for the genetic loci is conducted on a bead.

14. A method for strain-typing a target organism in a complex biological material, comprising the steps of:
a. amplifying nucleic acid sequences that contain variable genetic loci from the complex biological material, via a device, to generate an amplicon;
b. hybridizing the amplicon to one or more probes for the genetic loci, wherein the one or more probes will hybridize to a variant of the genetic loci and will not hybridize to a variant of the genetic loci, on at least one of a first hybridization array and a first bead;
c. detecting the one or more hybridized probes and the one or more non-hybridized probes on the at least one of the first hybridization array and the first bead;
d. assigning an identifier to each hybridized probe and non-hybridized probe on the at least one of the first hybridization array and the first bead;
e. generating a first pattern of one or more identifiers on the at least one of the first hybridization array and the first bead;
f comparing the first pattern of the at least one of the first hybridization array and the first bead to a second pattern of at least one of a second hybridization array and a second bead; and
g. determining if the first pattern is different from the second pattern.

15. The method of claim 14, wherein the first pattern and the second pattern are stored in a database operably connected to the device.

16. The method of claim 14, wherein the identifier is a binary digit.

17. The method of claim 14, further comprising the step of reporting the first pattern and the second pattern, wherein the first pattern and the second pattern represent a set of defining genetic characteristics of the complex biological material.

18. The method of claim 14, wherein there is a minimum of six genetic loci present in a target.

19. The method of claim 14, further comprising the step of capturing an image of the first hybridization array with a camera.

20. The method of claim 14, wherein the target is Listeria.
